(19) United States
(12) Patent Application Publication
(10) Pub. No.: US 2025/0312380 A1
(43) Pub. Date: Oct. 9, 2025

(54) COMPOSITIONS AND METHODS FOR ISOLATION OF MITOCHONDRIA FROM CRYOPRESERVED CELLS

(71) Applicant: The Sallie A. Burdine Breast Foundation, Baton Rouge, LA (US)

(72) Inventors: Xianpeng Jiang, Baton Rouge, LA (US); Brent M. Segal, Pembroke, MA (US); Catherine Baucom, Baton Rouge, LA (US)

(21) Appl. No.: 19/098,600

(22) Filed: Apr. 2, 2025

Related U.S. Application Data
(60) Provisional application No. 63/575,197, filed on Apr. 5, 2024.

(51) Int. Cl. A61K 35/28; C12N 5/00; C12N 5/0775

(52) U.S. Cl. CPC A61K 35/28; C12N 5/0662; C12N 5/525; C12N 5/562

(57) ABSTRACT

Described herein are methods, compositions, and kits for isolation of mitochondria from cryopreserved primary cells (e.g., fibroblast cells, mesenchymal stromal/stem cells) to be used in mitochondrial organelle transplantation (MOT) methods and compositions.

### CROSS REFERENCE TO RELATED APPLICATIONS

This application claims benefit of U.S. Provisional Patent Application No. 63/575, 197, filed Apr. 5, 2024, the text of which is incorporated herein by reference in its entirety.

### FIELD

The subject matter described herein relates to compositions, kits, and methods for the isolation of mitochondria from cryopreserved cells. In certain embodiments, the mitochondria thusly isolated are used for mitochondrial organelle transplantation in a subject.

### BACKGROUND

Mitochondria are organelles found within most eukaryotic cells. They serve an important function in cellular respiration and the generation of adenosine triphosphate (ATP). Mitochondrial organelle transplantation (MOT) was developed as a strategy for treatment of mitochondrial dysfunction or injury. Rapid treatment with MOT results in better efficacy in animal models of urgent diseases and injuries such as ischemic stroke, and traumatic brain and spinal cord injuries. However, current MOT procedures take several weeks to process cells from donors and obtain isolated mitochondrial compositions suitable for MOT. Furthermore, isolated mitochondria which are stored for long periods of time may experience functional declines, making them less suitable for use in MOT. Accordingly, there exists a need for improved mitochondrial organelle transplantation methods.

### SUMMARY

The present disclosure provides for methods, compositions, and kits for isolation of mitochondria from cryopreserved cells (e.g., primary cells, e.g., fibroblast cells, mesenchymal stromal/stem cells). Cryopreserved cells are a source of mitochondria which can be easily transported between and stored by medical facilities until mitochondria are needed for mitochondrial organelle transplantation (MOT). For example, primary cells may be obtained from a donor, expanded, and cryopreserved in a facility. Cryopreserved cells can be stored indefinitely in compact spaces with minimal maintenance requirements as compared to continuously cultured cells. Moreover, cryopreserved cells avoid phenotypic drift and infections, which continuously maintained cells are highly susceptible to. In certain embodiments, cryopreserved cells may then be transported to another facility or stored for later use. At another facility or at a later time, when needed, cryopreserved cells may then be thawed and mitochondria can be rapidly isolated from cells. Freshly isolated mitochondria may then be used to treat subjects who, for example, have experienced an injury and/or disease requiring MOT.

Additionally, as described herein, cryopreserved cells may be used to obtain healthy and robust mitochondria for rapid treatment of subjects suffering from conditions where outcomes are significantly improved by having readily available sources of mitochondria. As discussed above, cryopreserved cells have a number of advantages over continuously maintained cells. Furthermore, among other features, mitochondria isolated from cryopreserved cells have high adenosine triphosphate (ATP) content as compared to, for example, isolated mitochondria stored for long periods of time in a refrigerated buffer. As described herein, mitochondria obtained from primary cells, including mesenchymal stromal/stem cells (MSCs), have high ATP content, even after isolation from cryopreserved cells. Reliable sources of highly functioning isolated mitochondria are useful in treating diseases and injuries which require an urgent response including, for example, traumatic brain injuries, ischemic stroke, and spinal cord injuries.

In one aspect, the invention is directed to a method of isolating mitochondria from cryopreserved cells, the method comprising: thawing (e.g., a composition comprising) cryopreserved (i.e., frozen) primary cells; and isolating (e.g., directly isolating) the mitochondria from the thawed cells.

In certain embodiments, the method further comprises administering (e.g., a composition comprising) the isolated mitochondria to a subject (e.g., a human subject).

In certain embodiments, the primary cells are (e.g., are characterized as) mesenchymal stromal cells (MSCs). In certain embodiments, the MSCs have been obtained from bone marrow (e.g., of the iliac crest) of a human donor. In certain embodiments, the MSCs have been obtained from a member selected from the group consisting of adipose tissue, blood (e.g., peripheral blood), molar tissue (e.g., molar cells), a neonatal birth-associated tissue (e.g., placenta, umbilical cord), and a neonatal birth-associated fluid (e.g., amniotic fluid, umbilical cord blood).

In certain embodiments, the primary cells are (e.g., are characterized as) fibroblast cells. In certain embodiments, the fibroblast cells have been obtained from skin tissue of a donor (e.g., human foreskin fibroblasts).

In certain embodiments, the cryopreserved primary cells are stored in a cryopreservation media that does not comprise antibiotics.

In certain embodiments, the method comprises isolating the primary cells (e.g., MSCs, fibroblasts) (e.g., using flow cytometry) from tissue(s) or biological fluid(s) of a human donor.

In certain embodiments, method comprises isolating the primary cells (e.g., MSCs) from bone marrow (e.g., of the iliac crest) of the human donor.

In certain embodiments, the method comprises isolating the primary cells (e.g., MSCs) from a member selected from the group consisting of adipose tissue, blood (e.g., peripheral blood), molar tissue (e.g., molar cells), a neonatal birth-associated tissue (e.g., placenta, umbilical cord), and a neonatal birth-associated fluid (e.g., amniotic fluid, umbilical cord blood).

In certain embodiments, the method comprises isolating the primary cells (e.g., fibroblasts) from skin tissue.

In certain embodiments, the method comprises expanding the isolated cells (e.g., prior to cryopreservation).

In certain embodiments, the method comprises expanding the isolated cells for five passages or fewer (e.g., four or fewer passages, three or fewer passages, two or fewer passages, one passage) (e.g., prior to cryopreservation). For example, “expansion” of isolated cells means increasing the number of cells, e.g., through cell division. In certain embodiments, a “passage” refers to the process of removing cells from a culture vessel and transferring them to a new vessel with fresh growth medium to allow for continued expansion, e.g., and subculturing.

In certain embodiments, the method comprises expanding the isolated cells for about 2 population doublings to about 20 population doublings prior to cryopreservation (e.g., about 6 population doublings to about 10 population doublings) (e.g., fewer than 20 population doublings, fewer than 15 population doublings, fewer than 10 population doublings).

In certain embodiments, the method comprises inducing (e.g., stimulating) the isolated primary cells (e.g., stimulating the isolated cells during expansion) to increase an amount (e.g., a number of, a concentration of) of mitochondria within the isolated primary cells.

In certain embodiments, the method does not use antibiotics in the expansion of the isolated primary cells (e.g., wherein the media used to expand the isolated cells does not comprise antibiotics).

In certain embodiments, the method comprises cryopreservation of the isolated primary cells in a cryopreservation media. In certain embodiments, the cryopreservation media does not comprise antibiotics.

In certain embodiments, the method comprises identifying (e.g., characterizing, e.g., functionally characterizing) primary cells (e.g., MSCs, fibroblasts) (e.g., using flow cytometry) (e.g., prior to cryopreservation) having one or more characteristics (e.g., cell surface markers, immunomodulatory potential, cytokine secretions, methylation status) corresponding to a desired cell phenotype (e.g., amenable to mitochondrial isolation) (e.g., indicative of high-ATP content mitochondrial).

In certain embodiments, the one or more characteristics comprise one or more of (i) to (iv) as follows: (i) an absence and/or presence of (e.g., expression of) one or more cell surface marker(s), (ii) an immunomodulatory potential, (iii) a cytokine secretion (e.g., angiogenic cytokine secretion), and (iv) DNA methylation status.

In certain embodiments, the method comprises identifying (e.g., and selecting for) primary cells as expressing and/or presenting one or more (e.g., two or more, three or more, four or more) cell surface markers.

In certain embodiments, the one or more cell surface markers comprise one, two, three, or all four of the following cell surface markers: CD73, CD90, CD105, and CD166.

In certain embodiments, the method comprises identifying primary cells as not expressing and/or presenting one, two, three, four, or all five of the following cell surface markers: CD14, CD34, CD45, CD19, and HLA-DR.

In certain embodiments, the method comprises identifying primary cells comprising mitochondria characterized as having high adenosine triphosphate (ATP) content. In certain embodiments, the ATP content of the isolated mitochondria is greater than 24.5 pmol/mg mitochondria (e.g., greater than 26.3 pmol/mg mitochondria, greater than 30 pmol/mg mitochondria, greater than 35 pmol/mg mitochondria, greater than 40 pmol/mg mitochondria, greater than 44.1 pmol/mg mitochondria).

In certain embodiments, the method comprises selecting (e.g., using flow cytometry) the primary cells (e.g., prior to cryopreservation) based on, at least, one of the one or more characteristics corresponding to the desired cell phenotype.

In certain embodiments, the ATP content of the isolated mitochondria is greater than 24.5 pmol/mg mitochondria (e.g., greater than 26.3 pmol/mg mitochondria, greater than 30 pmol/mg mitochondria, greater than 35 pmol/mg mitochondria, greater than 40 pmol/mg mitochondria, or greater than 44.1 pmol/mg mitochondria).

In certain embodiments, the ATP of the isolated mitochondria obtained from the cryopreserved primary cells is substantially similar to the ATP content of mitochondria obtained from the primary cells prior to cryopreservation (e.g., within about 5%, within about 10%, within about 15%, within about 20%, within about 30% of the ATP content of isolated mitochondria from cells not subjected to cryopreservation).

In certain embodiments, the method comprises isolating the mitochondria from the thawed cells using differential centrifugation.

In certain embodiments, the method comprises suspending isolated mitochondria in a mitochondrial respiration buffer (MRB) (e.g., prior to administration). In certain embodiments, the mitochondrial respiration buffer (MRB) comprises: a buffering agent [e.g., a zwitterionic sulfonic acid buffering agent, e.g., 4-(2-hydroxyethyl)-1-piperazincethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose, e.g., sucrose at a concentration of about 240 mM); an agent that acts as a membrane stabilizer and/or oxygen radical scavenger and/or binder of Ca²⁺ and/or binder of free fatty acid (e.g., bovine serum albumin, BSA); and a serine protease inhibitor (e.g., phenylmethylsulfonyl fluoride (PMSF), also called phenylmethane sulfonyl fluoride).

In certain embodiments, the mitochondrial respiration buffer (MRB) does not comprise antibiotics.

In certain embodiments, the method comprises lysing the thawed primary cells (e.g., by homogenization, e.g., bead-beating).

In certain embodiments, the method does not comprise culturing and/or expanding the thawed primary cells.

In certain embodiments, the method comprises storing the isolated mitochondria (e.g., a composition comprising the isolated mitochondria) at a reduced temperature (e.g., at a temperature below 15° C., e.g., below 10° C., e.g., at a temperature within a range from about 0° C. to about 15° C., e.g., from about 1° C. to about 10° C., e.g., from about 2° C. to about 6° C.) (e.g., for at least one hour, at least 2 hours, at least 3 hours, at least about 6 hours, e.g., at least about 12 hours, e.g., at least about 24 hours, e.g., at least about 48 hours, e.g., at least about 5 days).

In certain embodiments, the cryopreserved primary cells are thawed (e.g., in a fluid bath, e.g., a water bath) at a temperature of about 20° C. to about 40° C. (e.g., about 37° C.).

In certain embodiments, the cryopreserved primary cells are stored at a temperature of about −60° C. or less (e.g., about −70° C. or less, about −80° C. or less, about −100° C. or less, about −120° C. or less, about −135° C. or less) (e.g., on dry ice, using liquid nitrogen).

In certain embodiments, the isolated mitochondria can be stored for at least one hour (e.g., at least 2 hours, at least 3 hours, at least about 6 hours, e.g., at least about 12 hours, e.g., at least about 24 hours, e.g., at least about 48 hours, e.g., at least about 5 days).

In certain embodiments, the isolated mitochondria are administered to the subject within 2 days of identification of any one or more of a condition (e.g., a disease, e.g., a disease related to mitochondrial dysfunction), an injury, and a symptom (e.g., a symptom related to mitochondrial dysfunction) (e.g., within 24 hours, within 12 hours, within 6 hours, within 3 hours, within 2 hours, or within 1 hour of said identification).

In certain embodiments, the subject has an acute condition (e.g., an acute injury, a sudden worsening and/or a sudden presentation of a disease, etc.).

In certain embodiments, the subject has a cardiac ischemic reperfusion injury, a traumatic brain injury (TBI) (e.g., mild TBI (mTBI)), a spinal cord injury, a cerebral stroke, a neurodegenerative disease, or any combination thereof.

In certain embodiments, the subject has lightheadedness, dizziness, blurred vision, tired eyes, ringing in the cars, a bad taste in the mouth, fatigue, lethargy, a change in sleep patterns, behavioral and/or mood changes, trouble with memory, concentration, attention, thinking, or any combination thereof.

In certain embodiments, the subject is being treated with a pharmaceutical agent (e.g., hydroxychloroquine and/or chloroquine) for indications accompanied by high Reactive Oxygen Species (ROS).

In certain embodiments, the method does not comprise administering to the subject an antibiotic.

In certain embodiments, the method further comprises administering to the subject a composition comprising one or more drugs and/or adjuvants.

In certain embodiments, the method comprises administering to the subject an iron-chelating agent (e.g., desferrioxamine or deferasirox).

In certain embodiments, the method comprising administering to the subject an antioxidant and/or a probiotic.

In certain embodiments, the method comprises administering to the subject a composition comprising the isolated mitochondria and a pharmaceutically acceptable carrier.

In certain embodiments, the method comprises adding the isolated mitochondria to a composition comprising extracellular vesicles (EVs) (e.g., after isolating the mitochondria) (e.g., prior to administration) to create an EV-mitochondria composition (e.g., for administration to a subject). In certain embodiments, the extracellular vesicles comprise one or more members selected from the group consisting of: (i) microvesicles (MVs) (e.g., ranging from about 100 nm to about 1 micrometer in diameter, e.g., comprising cytosolic and plasma membrane associated proteins), exosomes, and apoptotic bodies; (ii) microvesicles (MVs) (e.g., ranging from about 30 nm to about 150 nm in diameter, e.g., formed by an endosomal route); and (iii) apoptotic bodies (e.g., ranging from about 50 nm up to about 5 micrometers in diameter, e.g., comprising intact organelles and/or chromatin and/or glycosylated proteins).

In certain embodiments, the extracellular vesicles comprise extracellular vesicles of mesenchymal stromal cells (imEVs).

In certain embodiments, the EV-mitochondria composition comprises a mixture of mitochondria and EVs in a ratio from about 1:50 (mitochondria: EVs, in vol.) to about 50:1 (mitochondria: EVs, in vol.) [e.g., wherein the ratio is from about 2:1 to about 50:1, or wherein the ratio is from about 5:1 to about 15:1, or wherein the ratio is about 9:1].

In certain embodiments, the mitochondria accumulate within structures formed by the EVs in the EV-mitochondria composition.

In certain embodiments, the method improves preservation of mitochondrial membrane potential (MMP) of the isolated mitochondria (e.g., using extracellular vesicles to improve preservation of MMP of the isolated mitochondria and/or to improve preservation/retention of mitochondrial adenosine triphosphate (ATP) content) (e.g., using cryopreserved cells to improve preservation of MMP of the isolated mitochondria and/or to improve preservation/retention of mitochondrial adenosine triphosphate (ATP) content).

In certain embodiments, the method does not comprise use of antibiotics.

In certain embodiments, the method comprises isolating the mitochondria in an aseptic environment (e.g., an environment substantially free from contaminants, e.g., in a Xvivo System model X2).

In another aspect, the invention is directed to a kit comprising the isolated mitochondria produced by the methods described herein (e.g., as described above).

In another aspect, the invention is directed to a composition comprising the isolated mitochondria produced by the methods described herein (e.g., as described above).

In another aspect, the invention is directed to a composition (e.g., a pharmaceutical composition) comprising mitochondria (e.g., isolated mitochondria) characterized as having high (e.g., relatively high) ATP content (e.g., from cryopreserved MSCs) (e.g., a composition comprising a high concentration of mitochondria).

In certain embodiments, the ATP content of the mitochondria is greater than about 24.5 pmol/mg mitochondria (e.g., greater than 26.3 pmol/mg mitochondria, greater than 30 pmol/mg mitochondria, greater than 35 pmol/mg mitochondria, greater than 40 pmol/mg mitochondria, or greater than 44.1 pmol/mg mitochondria).

In certain embodiments, the composition comprises: a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose, e.g., sucrose at a concentration of about 240 mM); an agent that acts as a membrane stabilizer and/or oxygen radical scavenger and/or binder of Ca²⁺ and/or binder of free fatty acid (e.g., bovine serum albumin, BSA); and a serine protease inhibitor (e.g., phenylmethylsulfonyl fluoride (PMSF), also called phenylmethane sulfonyl fluoride).

In certain embodiments, the composition does not comprise antibiotics.

In certain embodiments, the mitochondria have been isolated from cryopreserved mesenchymal stromal cells (MSCs) (or cryopreserved cells characterized as MSCs).

In certain embodiments, the mitochondria have been isolated from cryopreserved fibroblasts (or cryopreserved cells characterized as fibroblasts).

In certain embodiments, the composition as described herein is for use in a method of treatment and/or prevention and/or amelioration of an injury (e.g., an acute injury) and/or a disease associated with mitochondrial damage.

In certain embodiments, the composition as described herein is for use in a method of treatment of a cardiac ischemic reperfusion injury, a traumatic brain injury (TBI) (e.g., mild TBI (mTBI)), a spinal cord injury, a cerebral stroke, a neurodegenerative disease, or any combination thereof.

In certain embodiments, the composition as described herein is for use in a method of treatment of lightheadedness, dizziness, blurred vision, tired eyes, ringing in the cars, a bad taste in the mouth, fatigue, lethargy, a change in sleep patterns, behavioral and/or mood changes, trouble with memory, concentration, attention, thinking, or any combination thereof.

In certain embodiments, the composition as described herein is for use in a method of treatment of a subject who is being treated with a pharmaceutical agent (e.g., hydroxychloroquine and/or chloroquine) for indications accompanied by high Reactive Oxygen Species (ROS).

In certain embodiments, the composition further comprises extracellular vesicles (EVs).

In certain embodiments, the extracellular vesicles comprise one or more members selected from the group consisting of: (i) microvesicles (MVs) (e.g., ranging from about 100 nm to about 1 micrometer in diameter, e.g., comprising cytosolic and plasma membrane associated proteins), exosomes, and apoptotic bodies; (ii) microvesicles (MVs) (e.g., ranging from about 30 nm to about 150 nm in diameter, e.g., formed by an endosomal route); and (iii) apoptotic bodies (e.g., ranging from about 50 nm up to about 5 micrometers in diameter, e.g., comprising intact organelles and/or chromatin and/or glycosylated proteins).

In certain embodiments, the extracellular vesicles comprise extracellular vesicles of mesenchymal stromal/stem cells (MSCs) (imEVs).

In certain embodiments, the composition comprises a mixture of mitochondria and EVs in a ratio from about 1:50 (mitochondria: EVs, in vol.) to about 50:1 (mitochondria: EVs, in vol.) [e.g., wherein the ratio is from about 2:1 to about 50:1, or wherein the ratio is from about 5:1 to about 15:1, or wherein the ratio is about 9:1].

In certain embodiments, the mitochondria accumulate within structures formed by the EVs.

In another aspect, the invention is directed to a kit comprising a composition as described herein (e.g., as described above).

In certain embodiments, the kit further comprises instructions for optimizing a dosage and/or frequency and/or route of administration of the composition.

In another aspect, the invention is directed to a method for transplantation of mitochondria in a subject for treatment and/or prevention and/or amelioration of an injury (e.g., an acute injury) and/or a condition associated with mitochondrial damage, the method comprising administering to said subject mitochondria isolated from cryopreserved primary cells (e.g., MSCs, fibroblast cells) of a donor to treat and/or prevent and/or ameliorate said injury and/or said condition.

In another aspect, the invention is directed to use of (e.g., a composition comprising) isolated donor mitochondria in the manufacture of a medicament for transplantation of mitochondria in a human subject, wherein the donor mitochondria are isolated from cryopreserved primary cells (e.g., MSCs, fibroblast cells) of a human donor, and wherein the isolated mitochondria are characterized as having a high (e.g., relatively high) ATP content.

In another aspect, the invention is directed to use of (e.g., a composition comprising) isolated donor mitochondria for transplantation of mitochondria in a human subject, wherein the donor mitochondria are isolated from cryopreserved primary cells of a human donor, and wherein the isolated mitochondria are characterized as having a high (e.g., relatively high) ATP content.

In another aspect, the invention is directed to isolated donor mitochondria (e.g., a composition comprising isolated donor mitochondria) for use in transplantation of mitochondria in a human subject, wherein the donor mitochondria are isolated from cryopreserved primary cells of a human donor, and wherein the isolated mitochondria are characterized as having a high (e.g., relatively high) ATP content.

In another aspect, the invention is directed to a kit comprising cryopreserved primary cells (e.g., an aqueous composition) in a sufficient quantity to obtain isolated mitochondria in a unit dosage effective to treat and/or prevent and/or ameliorate of an injury (e.g., an acute injury) and/or a disease associated with mitochondrial damage, the kit comprising: cryopreserved primary cells of a donor (e.g., MSCs, fibroblast cells); and a mitochondrial respiration buffer (MRB).

In certain embodiments, the cryopreserved primary cells are characterized in that the cells contain mitochondria having high ATP content.

In certain embodiments, the cryopreserved primary cells are preserved in a cryopreservation media that does not comprise an antibiotic.

In certain embodiments, the mitochondrial respiration buffer (MRB) comprises: one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)] [e.g., monopotassium phosphate (KH₂PO₄)]; a source of magnesium ion [e.g., magnesium chloride (MgCl₂)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an antioxidant (e.g., taurine); a cytoprotective agent that binds to calcium ion [e.g., lactobionate or salt thereof, e.g., K-lactobionate]; and an agent that acts as a membrane stabilizer and/or oxygen radical scavenger and/or binder of Ca²⁺ and/or binder of free fatty acid (e.g., bovine serum albumin, BSA).

In certain embodiments, the mitochondrial respiration buffer (MRB) does not comprise antibiotics.

In certain embodiments, the kit further comprises a mitochondrial isolation buffer (MIB).

In certain embodiments, the mitochondrial isolation buffer (MIB) comprises: a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminocthyl ether)-N,N,N′,N′-tetraacetic acid (EGTA) or salt thereof, e.g., K-EGTA)]; a sugar (e.g., sucrose); an agent that acts as a membrane stabilizer and/or oxygen radical scavenger and/or binder of Ca2+and/or binder of free fatty acid (e.g., bovine serum albumin, BSA); and a serine protease inhibitor (e.g., phenylmethylsulfonyl fluoride (PMSF), also called phenylmethane sulfonyl fluoride).

In certain embodiments, the mitochondrial isolation buffer (MIB) does not comprise antibiotics.

In certain embodiments, the kit further comprises extracellular vesicles (EVs).

In certain embodiments, the extracellular vesicles comprise one or more members selected from the group consisting of: (i) microvesicles (MVs) (e.g., ranging from about 100 nm to about 1 micrometer in diameter, e.g., comprising cytosolic and plasma membrane associated proteins), exosomes, and apoptotic bodies; (ii) microvesicles (MVs) (e.g., ranging from about 30 nm to about 150 nm in diameter, e.g., formed by an endosomal route); and (iii) apoptotic bodies (e.g., ranging from about 50 nm up to about 5 micrometers in diameter, e.g., comprising intact organelles and/or chromatin and/or glycosylated proteins).

In certain embodiments, the extracellular vesicles comprise extracellular vesicles of mesenchymal stromal/stem cells (MSCs) (imEVs).

In certain embodiments, the kit further comprises instructions for optimizing the dosage and/or frequency and/or route of administration of the isolated mitochondria.

In another aspect, the invention is directed to a method of isolating mitochondria for use in a method of treating a subject (e.g., a human subject), the method comprising: thawing cryopreserved (i.e., frozen) primary cells; and isolating mitochondria from the thawed cells, wherein the isolated mitochondria are characterized as having a high (e.g., relatively high) ATP content.

Any two or more of the features described in this specification, including in this summary section, may be combined to form implementations not specifically or explicitly described in this specification.

### DEFINITIONS

A or An: The articles “a” and “an” are used herein to refer to one or to more than one (i.e., to at least one) of the grammatical object of the article. By way of example, “an element” refers to one element or more than one element.

Administration: As used herein, the term “administration” typically refers to the administration of a composition to a subject or system, for example to achieve delivery of an agent that is, is included in, or is otherwise delivered by, the composition. Non-limiting examples of administration include oral administration; parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Agent: As used herein, the term “agent” refers to an entity (e.g., for example, a cell, a component of a cell such as mitochondria or other organelle, a small molecule, a peptide, a polypeptide, a nucleic acid, a lipid, a polysaccharide, a complex, a combination, a mixture, a system, or a phenomenon such as heat, electric current, electric field, magnetic force, magnetic field, etc.).

Allogeneic: As used herein, the term “allogeneic” refers to any material derived from one subject (a donor) and transplanted to another subject. Examples of allogeneic transplantation include allogeneic T cell transplantation, allogeneic stem cell transplantation, and, as discussed herein, allogeneic mitochondrial transplantation.

Amelioration: As used herein, the term “amelioration” refers to the prevention, reduction, palliation, or improvement of a state of a subject. Amelioration includes, but does not require, complete recovery or complete prevention of a disease, disorder or condition.

Antibiotic: As used herein, the term “antibiotic” refers to an antibacterial substance such as penicillin, gentamicin, streptomycin, cephalosporin, ciprofloxacin, or the like, that is used to treat or prevent infections by killing or inhibiting the growth of bacterial in or on the body, that is administered orally, topically, or by injection, and that is isolated from cultures of certain microorganisms (such as fungi) or is of semi-synthetic or synthetic origin.

Autologous: As used herein, the term “autologous” refers to any material derived from one subject (a donor) and transplanted back into that same subject. Herein “autologous” and “autogenic” are used interchangeably. Examples of autologous transplantation include autologous stem cell transplantation and autologous stem cell transplantation, and, as discussed herein, autologous mitochondrial transplantation.

Biological Sample: As used herein, the term “biological sample” typically refers to a sample obtained or derived from a biological source (e.g., a tissue or organism or cell culture) of interest, as described herein. In some embodiments, e.g., as set forth herein, a biological source is or includes an organism, such as an animal or human. In some embodiments, e.g., as set forth herein, a biological sample is or include biological tissue or fluid. In some embodiments, e.g., as set forth herein, a biological sample can be or include cells, tissue (e.g., skin tissue, muscle, or other tissue), or bodily fluid (e.g., “biological fluid”). In some embodiments, e.g., as set forth herein, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or bronchioalveolar lavages, aspirates, scrapings, or bone marrow. In some embodiments, e.g., as set forth herein, a biological sample is or includes cells obtained from a single subject or from a plurality of subjects. A sample can be a “primary sample” obtained directly from a biological source or can be a “processed sample.” A biological sample can also be referred to as a “sample.”

Improved, increased, or reduced: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.

Isolated: As used herein, “isolated” refers to a substance and/or entity (e.g., including one or more mitochondria) that has been (a) separated from at least some of the components with which it was associated when initially produced (whether in nature, in a subject such as a donor, and/or in an experimental setting), and/or (b) designed, produced, prepared, and/or manufactured by the hand of man. Isolated substances and/or entities may be separated from at least about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated substances and/or entities are at least about 80%, about 85%, about 90%, about 91%, about 92%, about 93%, about 94%, about 95%, about 96%, about 97%, about 98%, about 99%, or more than about 99% pure. As used herein, a substance and/or entity is “pure” if it is substantially free of other components. In some embodiments, as will be understood by those skilled in the art, a substance and/or entity may still be considered “isolated” or “pure” after having been combined with certain other components such as, for example, one or more carriers or excipients (e.g., buffer, solvent, water, etc.); in such embodiments, percent isolation or purity of the substance and/or entity is calculated without including such carriers or excipients. To give but one example, in some embodiments, mitochondria that occur in nature can be referred to as “isolated” when, (a) they are present in a composition that does not include some or all of the components with which they were associated in nature, e.g., in a donor from which they were derived; (b) they are substantially free of other organelles of a donor organism from which they were derived; (c) they are present in a cell or system that differs from the donor organism from which they were derived. Thus, for instance, mitochondria removed from a donor for transplantation into a second different subject can be referred to as “isolated.”

Neurodegenerative disease: As used herein, the term “neurodegenerative disease” (also referred to as “degenerative nerve disease”) is an umbrella term for conditions which primarily affect the neurons in the human brain. In certain instances, neurodegenerative disease is characterized by a progressive loss of neurons associated with deposition of proteins showing altered physicochemical properties in the brain and/or in peripheral organs. Neurodegenerative diseases include, for example, amyotrophic lateral sclerosis (ALS), Parkinson's disease (PD) and PD-related disorders, Alzheimer's disease (AD), Lewy body dementia (LBD), other forms of dementia, muscular dystrophy (MD), mitochondrial disorders, prion diseases, motor neuron diseases (MND), Huntington's disease (HD), multiple sclerosis (MS), spinocerebellar ataxia (SCA), spinal muscular atrophy (SMA), Friedreich's ataxia, Batten disease, fatal familial insomnia, and others.

Pharmaceutical composition: As used herein, the term “pharmaceutical composition” refers to a composition in which an active agent (e.g., “pharmaceutical agent”) is provided together with one or more pharmaceutically acceptable carriers. In some embodiments, e.g., as set forth herein, the active agent is present in a unit dose amount appropriate for administration to a subject, e.g., in a therapeutic regimen that shows a statistically significant probability of achieving a predetermined therapeutic effect when administered to a relevant population. In some embodiments, e.g., as set forth herein, a pharmaceutical composition can be formulated for administration in a particular form (e.g., in a solid form or a liquid form), and/or can be specifically adapted for, for example: oral administration (for example, as a drench (aqueous or non-aqueous solutions or suspensions), tablet, capsule, bolus, powder, granule, paste, etc., which can be formulated specifically for example for buccal, sublingual, or systemic absorption); parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation and/or modifies bioavailability of an agent, e.g., a pharmaceutical agent. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.

Primary Cells: As used herein, in certain embodiments, the term “primary cells” refers to cells that are isolated directly from a living organism (e.g., a tissue or organ) and have not undergone an immortalization process. Primary cells are distinguished from established cell lines (which are derived from primary cells) in that primary cells generally have a finite life span. Fibroblasts and mesenchymal stem cells (MSCs) (also known as mesenchymal stromal cells) may be considered primary cells as they may be isolated directly from tissues and are not derived from cell lines.

Prognosis: As used herein, the term “prognosis” refers to determining the qualitative or quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.

Reference: As used herein describes a standard or control relative to which a comparison is performed. For example, in some embodiments, e.g., as set forth herein, an agent, subject, animal, individual, population, sample, sequence, or value of interest is compared with a reference or control agent, subject, animal, individual, population, sample, sequence, or value. In some embodiments, e.g., as set forth herein, a reference or characteristic thereof is tested and/or determined substantially simultaneously with the testing or determination of the characteristic in a sample of interest. In some embodiments, e.g., as set forth herein, a reference is a historical reference, optionally embodied in a tangible medium. Typically, as would be understood by those of skill in the art, a reference is determined or characterized under comparable conditions or circumstances to those under assessment, e.g., with regard to a sample. Those skilled in the art will appreciate when sufficient similarities are present to justify reliance on and/or comparison to a particular possible reference or control.

Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample).

Susceptible to: An individual who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status associated with, development of the disease, disorder, or condition. In some embodiments, e.g., as set forth herein, a risk of developing a disease, disorder, and/or condition is a population-based risk (e.g., family members of individuals suffering from the disease, disorder, or condition).

Subject: As used herein, the term “subject” refers to an organism, typically a mammal (e.g., a human). In some embodiments, e.g., as set forth herein, a subject is suffering from a disease, disorder or condition. In some embodiments, e.g., as set forth herein, a subject is susceptible to a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”

Syngeneic: As used herein, the term “syngeneic” refers to any material derived from one subject (a donor) and transplanted to a subject wherein the subject and donor are genetically identical. Examples of syngeneic transplantation include syngeneic T cell transplantation, syngeneic bone marrow transplantation, and, as discussed herein, syngeneic mitochondrial transplantation.

Therapeutic agent, pharmaceutical agent, and active agent: As used herein, the terms “therapeutic agent”, “pharmaceutical agent”, and “active agent” are interchangeable, and each refers to any agent that elicits a desired pharmacological effect when administered to a subject. In some embodiments, e.g., as set forth herein, an agent is considered to be a therapeutic agent if it demonstrates a statistically significant effect across an appropriate population. In some embodiments, e.g., as set forth herein, the appropriate population can be a population of model organisms or a human population. In some embodiments, e.g., as set forth herein, an appropriate population can be defined by various criteria, such as a certain age group, gender, genetic background, preexisting clinical conditions, etc. In some embodiments, e.g., as set forth herein, a therapeutic agent is a substance that can be used for treatment of a disease, disorder, or condition. In some embodiments, e.g., as set forth herein, a therapeutic agent is an agent that has been or is required to be approved by a government agency before it can be marketed for administration to humans. In some embodiments, e.g., as set forth herein, a therapeutic agent is an agent for which a medical prescription is required for administration to humans.

Therapeutically effective amount: As used herein, the term “therapeutically effective amount” refers to an amount that produces a desired effect for which it is administered. In some embodiments, e.g., as set forth herein, the term refers to an amount that is sufficient, when administered to a population suffering from or susceptible to a disease, disorder, or condition, in accordance with a therapeutic dosing regimen, to treat the disease, disorder, or condition. Those of ordinary skill in the art will appreciate that the term therapeutically effective amount does not in fact require successful treatment be achieved in a particular individual. Rather, a therapeutically effective amount can be an amount that provides a particular desired pharmacological response in a significant number of subjects when administered to individuals in need of such treatment. In some embodiments, e.g., as set forth herein, reference to a therapeutically effective amount can be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent can be formulated and/or administered in a single dose. In some embodiments, e.g., as set forth herein, a therapeutically effective agent can be formulated and/or administered in a plurality of doses, for example, as part of a multi-dose dosing regimen.

Treatment: As used herein, the term “treatment” (also “treat” or “treating”) refers to administration of a therapy that partially or completely alleviates, ameliorates, relieves, inhibits, delays onset of, halts progression of, slows progression of, reverses progression of, reduces severity of, and/or reduces incidence of one or more symptoms, features, and/or causes of a particular disease, disorder, or condition, or is administered for the purpose of achieving any such result. In some embodiments, e.g., as set forth herein, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, e.g., as set forth herein, treatment can be of a subject who has been diagnosed as suffering from the relevant disease, disorder, and/or condition. In some embodiments, e.g., as set forth herein, treatment can be of a subject known to have one or more susceptibility factors that are statistically correlated with increased risk of development of the relevant disease, disorder, or condition. In various examples, treatment is of a cancer.

Unit dose: As used herein, the term “unit dose” refers to an amount administered as a single dose and/or in a physically discrete unit of a pharmaceutical composition. In many embodiments, e.g., as set forth herein, a unit dose contains a predetermined quantity of an active agent. In some embodiments, e.g., as set forth herein, a unit dose contains an entire single dose of the agent. In some embodiments, e.g., as set forth herein, more than one-unit dose is administered to achieve a total single dose. In some embodiments, e.g., as set forth herein, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic agent(s). For example, acceptable carriers (e.g., pharmaceutically acceptable carriers), diluents, stabilizers, buffers, preservatives, etc., can be included. It will be appreciated by those skilled in the art, in many embodiments, e.g., as set forth herein, a total appropriate daily dosage of a particular therapeutic agent can comprise a portion, or a plurality, of unit doses, and can be decided, for example, by a medical practitioner within the scope of sound medical judgment. In some embodiments, e.g., as set forth herein, the specific effective dose level for any particular subject or organism can depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts

### BRIEF DESCRIPTION OF THE DRAWINGS

Drawings are presented herein for illustration purposes, not for limitation. The foregoing and other objects, aspects, features, and advantages of the present disclosure will become more apparent and better understood by referring to the following description taken in conjunction with the accompanying drawings, in which:

FIG. 1 is a series of fluorescent images (FIG. 1, panels a and b) comparing the mitochondrial membrane potential (MMP) of mitochondria from fresh fibroblast (FIG. 1, panel a) with cryopreserved fibroblasts (FIG. 1, panel b), according to an illustrative embodiment.

FIG. 2 is a series of fluorescent images (FIG. 2, panels a and b) comparing the mitochondrial membrane potential (MMP) of mitochondria from cryopreserved MSCs (FIG. 2, panel b) to fresh MSCs (FIG. 2, panel a), according to an illustrative embodiment.

FIG. 3 shows a series of fluorescent and phase contrast images comparing mitochondria of cryopreserved MSCs and fresh MSCs, according to an illustrative embodiment. FIG. 3, panel a is a phase contrast image of mitochondria from fresh MSCs. FIG. 3, panel b is a fluorescent image of mitochondria of fresh MSCs. FIG. 3, panel c is a phase contrast image of mitochondria from cryopreserved MSCs. FIG. 3, panel d is a fluorescent image of mitochondria of cryopreserved MSCs.

FIG. 4 shows a series of images of mitochondria isolated from fresh and frozen MSCs transferred into NSC-34 cells, according to an illustrative embodiment. FIG. 4, panels a and b show phase contrast and fluorescent images, respectively, of NSC-34 cells co-cultured with the mitochondria of fresh MSCs. FIG. 4, panels c and d show phase contrast and fluorescent images of NSC-34 cells, respectively, co-cultured with the mitochondria of cryopreserved MSCs.

FIG. 5 shows a series of phase contrast images of fibroblast cells (FIG. 5, panel a) and MSCs (FIG. 5, panel b), according to an illustrative embodiment.

FIG. 6 shows a graph of ATP content and cell number of fibroblasts and MSCs, according to an illustrative embodiment.

FIG. 7 shows a graph of JC-1 aggregates of mitochondria isolated from fibroblasts and MSCs, according to an illustrative embodiment.

FIG. 8 shows a graph of ATP content of mitochondria isolated from fibroblasts and MSCs, according to an illustrative embodiment.

FIG. 9 shows an illustrative flow diagram of an exemplary method described herein.

### DESCRIPTION OF THE INVENTION

Incorporated herein by reference is International (PCT) Patent Application No. PCT/US2020/047359, filed Aug. 21, 2020, and published as International Publication No. WO 2021/141637, which describes compositions and methods for treatment of amyotrophic lateral sclerosis (ALS); other neurodegenerative diseases (ND) such as Alzheimer's disease, Parkinson's disease, muscular dystrophy; and other mitochondrial disorders. In particular, described therein are experimental examples demonstrating mitochondrial organelle transplantation (MOT™) for the treatment of NDs such as ALS. Also incorporated herein is International (PCT) Patent Application No. PCT/US23/32292, filed Sep. 8, 2023, which describes compositions and methods for treatment of traumatic brain injury (TBI), for example, mild traumatic brain injury (mTBI). Also incorporated herein is International (PCT) Patent Application No. PCT/US23/32294, filed Sep. 8, 2023, which describes compositions and methods for treatment of spaceflight-associated mitochondrial damage. Also, incorporated herein is U.S. Provisional Application No. 63/455,397, filed Mar. 29, 2023, U.S. Provisional Application No. 63/604,044, filed Nov. 29, 2023, and International (PCT) Patent Application No. PCT/US24/22014, filed Mar. 28, 2024, which describe therein experimental examples of compositions and methods for the preservation of isolated mitochondria. The contents of each of the above-referenced patent applications are incorporated by reference herein in their entirety. Moreover, all publications mentioned herein are incorporated by reference herein in their entirety.

It is contemplated that compositions, methods, and processes of the claimed invention encompass variations and adaptations developed using information from the embodiments described herein. Adaptation and/or modification of the compositions, methods, and processes described herein may be performed, as contemplated by this description.

Throughout the description, where compositions, articles, devices, and systems are described as having, including, or comprising specific components, or where processes and methods are described as having, including, or comprising specific steps, it is contemplated that, additionally, there are compositions, articles, devices, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein.

Documents are incorporated herein by reference as noted. Where there is any discrepancy in the meaning of a particular term, the meaning provided in this document is controlling.

Headers are provided for the convenience of the reader-the presence and/or placement of a header is not intended to limit the scope of the subject matter described herein.

Mitochondria are organelles of eukaryotic cells that function to produce adenosine triphosphate (ATP) by oxidative phosphorylation (OXPHOS) in the presence of oxygen. They are also associated with the synthesis of iron-sulfur clusters and heme, β-oxidation of fatty acids, and homeostasis of calcium, iron and reactive oxygen species (ROS). Mitochondrial dysfunction plays an important role in many diseases such as cardiovascular disease, metabolic disease, neurodegenerative disease, ischemic reperfusion injuries, and traumatic brain and spinal cord injuries, among other diseases and injuries.

In recent years, mitochondrial organelle transplantation (MOT) has been used as a therapeutic intervention that benefits neuronal survival and regeneration for neurodegenerative diseases, ischemic reperfusion injuries, traumatic brain injuries, and spinal cord injuries, among other conditions.

Masuzawa A et al (2013) have studied the role of injected isolated mitochondria for cardio protection during ischemia-reperfusion. The exogenous mitochondria can enter into cardiomyocytes within 2 hours after injection and maintain viability and function producing adequate ATP levels. They also demonstrated that the exogenous mitochondria provided cardio protection both extracellularly and intracellularly (Masuzawa A, et al Transplantation of autologously derived mitochondrial protects the heart from ischemia-reperfusion injury. (Am. J. Physiology-Heart and Circulatory Physiology, 2012; 304: H966-H982. https://doi.org/10.1152/ajpheart.00883.2012.).

Huang and colleagues (2016) demonstrated that local intracerebral or systemic intra-arterial injection of isolated hamster mitochondria into brain-ischemic rats significantly reduced neuronal death and restored motor performance. They found that the mitochondrial internalization to neurons could not completely account for the high rescue of neuronal injury. Extracellularly exogenous mitochondria may be a source of ATP and a ROS scavenger to protect cells from damage by free radicals (Huang PJ, et al. Transferring xenogenic mitochondria provides neural protection against ischemic stress in ischemic rat brains. (Cell Transplantation 2016; 25:913-927. https://doi.org/10.3727/096368915X689785). Transplantation of placenta-derived mitochondria via intravenous infusion significantly decreased brain infarction after focal cerebral ischemia in mice. In animal experiments of traumatic brain and spinal cord injury, transplantation of allogeneic mitochondria at the early stage of spinal cord injury (SCI) reduces mitochondrial fragmentation, neuro-apoptosis, neuroinflammation, and generation of oxidative stress, thus leading to improved functional recovery following traumatic SCI. Mitochondria transplantation also significantly reduced neuronal death and memory impairment following traumatic brain injury (TBI).

The sooner a patient is treated with MOT, the better efficacy MOT has in treating acute and/or urgent conditions (e.g., injuries and/or diseases). Typical MOT procedures require cell expansion for several weeks, followed by mitochondrial isolation and subsequent patient injection. Accordingly. routine MOT procedures are not ideal for usage in treating acute and/or urgent conditions including, but not limited to, ischemic-reperfusion stroke, traumatic brain, and spinal cord injuries.

Human cells can be kept in liquid nitrogen for long-term storage. If cryopreserved cells in cell banks provide viable intact mitochondria, MOT could be useful for urgent treatment of diseases and conditions. Accordingly, the inventors isolated mitochondria from cryopreserved human fibroblast cells and mesenchymal stromal/stem cells (MSCs) from cell banks, and compared the mitochondrial viability and transfer ability with the mitochondria from fresh cells. In the present application, the terms mesenchymal stem cells (MSCs) and mesenchymal stromal cells are used interchangeably. Applicant obtained results demonstrating that mitochondria from MSCs had more ATP content than mitochondria from fibroblasts.

### A. Mitochondria

In eukaryotic cells, mitochondria are known as the powerhouse, which generates adenosine triphosphate (ATP), by oxidative phosphorylation (OXPHOS). They also play an important role in synthesis of iron-sulfur clusters and heme, β-oxidation of fatty acids, homeostasis of calcium, iron and reactive oxygen species (ROS). Mitochondria are of particular importance in neurons. Neurons have high metabolic requirements—the brain consumes 20% of the body's resting ATP production despite being only 2% of its mass. Moreover, mitochondria are essential calcium buffering organelles in neurons that modulate local calcium dynamics, for example, regulate neurotransmitter release. Neurons are long-lived cells that persist throughout the lifespan of the individual and as such are more susceptible to the accumulating damage arising from mitochondrial dysfunction. Severe mitochondrial dysfunction comes in many forms, including defective OXPHOS, excessive ROS, impaired calcium buffering capacity, and defective mitochondrial dynamics.

In eukaryotic cells, mitochondria generate ATP by oxidative phosphorylation (OXPHOS) in the presence of oxygen. Mitochondria also play an important role in synthesis of iron-sulfur (Fe—S) clusters, β-oxidation of fatty acids, synthesis of heme prosthetic groups, the urea cycle, as well as homeostasis of calcium, iron and reactive oxygen species (ROS). Mitochondria are highly dynamic organelles which frequently fuse and divide. Mitochondrial fusion/fission allow segregation of damaged mitochondria, mitophagy to remove damaged mitochondria, and ultimately cell death if the damage is too severe. In addition, mitochondria can transfer between cells. Cells may be able to obtain functional mitochondria from other cells in order to satisfy their bioenergetics and biosynthetic needs. Without wishing to be bound to any particular theory, the possible mechanisms include tunneling nanotubes, extracellular vesicles and partial or complete cell fusion.

Mitochondrial dysfunction contributes to many diseases such as neurodegenerative disease, cardiac disease, and cancer. Mitochondrial dysfunction broadly includes states in which mitochondria of a cell, tissue, organism, or sample thereof, are characterized by (1) a decreased rate, amount, or efficiency of ATP production; (2) a decreased mitochondrial membrane potential; (3) a decreased number or concentration of mitochondria; and/or (4) an increased rate or amount of ROS production, relative to a reference. Methods and techniques for measuring mitochondrial ATP production, include among other things, mitochondrial membrane potential, number or concentration of mitochondria, and/or ROS production.

Mitochondrial dysfunction has been documented in amyotrophic lateral sclerosis (ALS), Alzheimer's disease (AD), and Parkinson's disease (PD), for example. Mitochondria are essential for neural function because neurons highly depend on aerobic OXPHOS in mitochondria for their energetic needs. Defective mitochondrial respiration and ATP production in neurons result in neural dysfunction and degeneration. Mitochondria also produce ROS. If oxidative stress of ROS overwhelms the antioxidative defense most from superoxide dismutase (SOD), catalase (CAT) and glutathione peroxidase (GPX), ROS causes protein, lipid and DNA damage of neurons. In addition, overload of mitochondrial calcium and iron impairs ATP production and structures of mitochondria and neurons.

Mitochondria are highly present in cardiac cells due to the increased energy demands of such cells. Mitochondrial dysfunction is associated with the development of cardiac diseases including as atherosclerosis, ischemia-reperfusion injury, hypertension, cardiac hypertrophy, and heart failure.

Defects in mitochondrial function have also been linked to tumorigenesis. For example, it has been observed that cancer cells have an increase in glycolysis and lactate production in the presence of oxygen without an increase in OXPHOS, known as the “Warburg Effect”. Many cancers have mitochondrial defects and dysfunction. Glycolytic inhibitors have been found to suppress tumor growth in animal models and clinical trials.

In certain embodiments, the methods and compositions described herein are used in treatment, prevention, or amelioration of injuries (e.g., acute injuries) and/or a disease associated with mitochondrial damage (e.g., mitochondrial dysfunction). Early intervention through administering a therapeutically effective amount of mitochondria may be able to treat, prevent, or ameliorate injuries and/or diseases when provided to a subject quickly after the subject experiences the condition. In certain embodiments, isolated mitochondria can be stored for at least 1 hour (e.g., at least 2 hours, at least 6 hours, at least 12 hours, at least 24 hours, at least 2 days) prior to administration to a subject suffering from an injury and/or a disease. In some embodiments, the isolated mitochondria are administered to the subject within 2 days of identification of a condition (e.g., a disease, e.g., a disease related to mitochondrial dysfunction), an injury, and/or a symptom (e.g., a symptom related to mitochondrial dysfunction) (e.g., within 24 hours, within 12 hours, within 6 hours, within 3 hours, within 2 hours, or within 1 hour of said identification).

### B. Treatment of Traumatic Brain Injury

Methods and compositions herein may be used to treat, prevent, or ameliorate acute injuries including traumatic brain injury (TBI) or symptoms related to a traumatic brain injury (TBI). Without wishing to be bound to any particular theory, a TBI can cause damage to and/or loss of mitochondria, which can result in secondary injuries to the brain. Secondary injuries due to mitochondrial damage (e.g., dysfunction) or loss can cause symptoms related to a TBI. Accordingly, among other things, administering MOT to a subject quickly after an injury is identified is important to prevent long-term damage.

In certain embodiments, a TBI is as caused by a mechanical injury to the head from an external force (e.g., a fall, an explosion, a car accident, a shockwave, or other mechanical force). In certain embodiments, a mechanical injury to the head includes a bump, blow, and/or jolt to the head, and/or penetrating head injury.

In certain embodiments, a TBI can result in one or more symptoms including, but not limited to, a headache (e.g., that progressively gets worse and/or does not go away), repeated vomiting, nausea, convulsions (e.g., seizures), an inability to awaken from sleep, dilation of one or both pupils of the eyes, slurred speech, weakness and/or numbness in the extremities, loss of coordination, increased confusion, restlessness, and agitation.

In certain embodiments, a TBI is a mild traumatic brain injury (mTBI) (e.g., a concussion). In certain embodiments, an mTBI can result in one or more symptoms including, but not limited to, lightheadedness, dizziness, blurred vision, tired eyes, ringing in the cars, a bad taste in the mouth, fatigue, lethargy, a change in sleep patterns, behavioral and/or mood changes, and trouble with memory, concentration, attention, and/or thinking.

### C. Selection, Characterization, and Isolation of Cells

In certain embodiments, cells used in the methods and compositions herein are primary cells. In certain embodiments, cells used in the methods, kits, and compositions described herein are fibroblast cells or MSCs obtained from a mammalian donor (e.g., a human donor).

In certain embodiments, primary MSCs are isolated from a suitable solid tissue and/or a biological fluid of a donor (e.g., a human donor). In certain embodiments, MSCs are isolated from bone marrow, adipose tissue, blood (e.g., peripheral blood), molar tissue (e.g., molar cells), neonatal birth-associated tissues or fluids such as placenta, umbilical cord, amniotic fluid, or umbilical cord blood. In certain embodiments, MSCs are obtained from the iliac crest.

In certain embodiments, fibroblasts are obtained from human skin tissues (e.g., human foreskin fibroblasts).

In certain embodiments, primary cells used to obtain isolated mitochondria are identified (e.g., characterized) as having one or more characteristics associated with (corresponding to) a desired cell type as determined by cell phenotype. For example, in certain embodiments, an absence and/or presence of (e.g., expression of) one or more cell surface marker(s), immunomodulatory potential of the cells, cytokine secretion(s) (e.g., angiogenic cytokine secretion), or DNA methylation status can be used to identify a desired cell type. For example, MSCs can be identified by detecting the presence of one or more cell surface markers (e.g., two or more, three or more, four or more) cell surface markers. For example, cells expressing one, two, three, or all four of the cell surface markers CD73, CD90, CD105, and CD166 may be characterized as MSCs. In some embodiments, MSCs and fibroblast cells do not express one, two, three, four, or all five of the following cell surface markers CD14, CD34, CD45, CD19, and HLA-DR. In some embodiments, cells expressing one, two, or all three of the cell surface markers CD73, CD90, CD105 may be characterized as MSCs or fibroblasts. In some embodiments, the combination of markers that cells express or present and markers that cells do not express or present are used to identify cells. In some embodiments, cells can be selected on the basis of their surface markers using one or more techniques available in the art. For example, flow cytometry can be used to select cells isolated from donor tissues and/or fluids to obtain substantially pure populations of a desired cell type.

In some embodiments, primary cells used to obtain isolated mitochondria are identified using DNA methylation status. Methods of determining methylation status of methylation loci are known in the art. In some embodiments, hypermethylation and/or hypomethylation of one or more methylation loci (e.g., regions, genes, individual CpG sites) may be used to identify cells (e.g., MSCs, fibroblasts). In some embodiments, methylation status may include a number (e.g., an amount) of methylated loci, a frequency of methylated loci (e.g., within a region), or a pattern of methylated loci (e.g., hypermethylation or hypomethylation of one or more regions).

In some embodiments, primary cells obtained from a donor undergo expansion prior to cryopreservation. Expansion allows for the multiplication of an initial cell population. In some embodiments, the number of population doublings which cells undergo prior to cryopreservation are limited in order to prevent or limit phenotypic drift and cell aging. Without wishing to be bound to any particular theory, MSCs are susceptible to phenotypically drifting over time as they undergo population doublings. In some embodiments, cells are expanded for about 2 to about 20 population doublings prior to cryopreservation (e.g., about 6 population doublings to about 10 population doublings) (e.g., fewer than 20 population doublings, fewer than 15 population doublings, fewer than 10 population doublings). In some embodiments, cell passage number is used to measure cell age. In some embodiments, isolated cells are expanded for five passages or fewer (e.g., four or fewer passages, three or fewer passages, two or fewer passages, one passage) prior to cryopreservation.

In certain embodiments, a donor from which tissues and primary cells are obtained is selected as required by FDA 21 CFR Part 1271, which is incorporated by reference in its entirety. In certain embodiments, a donor is screened for one or more diseases (e.g., communicable diseases, e.g., a virus). In certain embodiments, a donor is free from risk factors for, and clinical evidence of, infection due to a relevant communicable disease agent and/or disease. In certain embodiments, a donor is free from communicable disease risks associated with xenotransplantation. In certain embodiments, a donor is tested for communicable disease agents and is determined to be negative or nonreactive. In certain embodiments, a donor is free from diseases including, but not limited to, human immunodeficiency virus (HIV), hepatitis B virus, hepatitis C virus, human transmissible spongiform encephalopathy (e.g., Creutzfeldt-Jakob disease), treponema pallidum, and communicable diseases of the genitourinary tract (e.g., chlamydia trachomatis, neisseria gonorrhea).

In certain embodiments, a donor does not have a neurodegenerative disease and/or other condition associated with mitochondrial dysfunction. In certain embodiments, a donor does not have a disease including, but not limited to, amyotrophic lateral sclerosis (ALS), Parkinson's disease (PD), a PD related disorder, Alzheimer's disease (AD), Lewy body dementia (LBD), dementia, muscular dystrophy (MD), a mitochondrial disorder, prion disease, motor neurone disease (MND), Huntington's disease (HD), multiple sclerosis (MS), spinocerebellar ataxia (SCA), spinal muscular atrophy (SMA), Friedreich's ataxia, Batten disease, and fatal familial insomnia.

As is disclosed herein, in various embodiments, a mitochondrial donor and a subject of mitochondrial transplantation or treatment do not need to be an HLA (human leukocyte antigen) match [e.g., not an identical match (e.g., based on match of 8 or 10 tested HLA markers) and/or not a haploidentical match (e.g., based on match of 8 or 10 tested HLA markers), and/or of indeterminate match status (e.g., no HLA markers tested prior to the administering step). Humans have three main MHC class I loci, known as HLA-A, HLA-B, and HLAC, each individual carrying two alleles at each locus. Humans have six main MHC class II loci, known as HLA-DPA1, HLA-DPB1, HLA-DQA1, HLA-DQB1, HLA-DRA, and HLA-DRB1, each individual carrying two alleles at each locus. In general, a donor and a subject would be matched based on alleles present in the donor and the subject at one or more HLA loci, such as HLA-A, HLA-B, HLA-C, HLA-DRB1, HLA-DQB1, and/or HLA-DPB1. Various standards for HLA matching are known in the art. Matching of all 8 alleles at HLA-A, HLA-B, HLA-C, and HLA-DRB1 loci can be referred to as an 8/8 match. Matching of all 10 alleles at HLA-A, HLAB, HLA-C, HLA-DRB1, and HLA-DQB1 loci can be referred to as a 10/10 match. For certain transplantations, varying degrees of allele mismatch can be accepted. Thus, for example, a donor and a subject can be matched, e.g., at 10, 9, 8, 7, 6, 5, 4, 3, 2, 1, or 0 alleles of 10 at HLA-A, HLA-B, HLA-C, HLA-DRB1, and HLA-DQB1, or at 8, 7, 6, 5, 4, 3, 2, 1, or 0 alleles of 8 HLAA, HLA-B, HLA-C, and HLA-DRB1. This is a significant advantage over stem cell therapy and organ transplantation which require HLA matching of donor and recipient.

### D. Isolated Mitochondria & Mot

Among other things, methods, compositions, and kits described herein are used to isolate mitochondria for MOT. Mitochondria isolated from cryopreserved cells can be evaluated for function by measuring mitochondrial ATP production, mitochondrial membrane potential (MMP), number or concentration of mitochondria, and ROS production.

In certain embodiments, mitochondria isolated from cryopreserved cells (e.g., primary cells) as described herein are characterized as having high ATP content. In certain embodiments, the ATP content of isolated mitochondria is greater than 24.5 pmol/mg mitochondria (e.g., greater than 26.3 pmol/mg mitochondria, greater than 30 pmol/mg mitochondria, greater than 35 pmol/mg mitochondria, greater than 40 pmol/mg mitochondria, greater than 44.1 pmol/mg mitochondria). In certain embodiments, the ATP of isolated mitochondria obtained from cryopreserved primary cells is substantially similar to the ATP content of mitochondria obtained from the primary cells prior to cryopreservation (e.g., within about 5%, within about 10%, within about 15%, within about 20%, within about 30% of the ATP content of isolated mitochondria from cells not subjected to cryopreservation).

In certain embodiments, mitochondria isolated from cryopreserved cells have comparable respiratory capacity and mitochondrial membrane structures to mitochondria isolated from primary cells which have not been cryopreserved.

In certain embodiments, methods and compositions described herein improve preservation of mitochondrial membrane potential (MMP) of isolated mitochondria. For example, storing mitochondria for excessive periods of time results in the loss of MMP, while freshly isolated mitochondria maintains MMP.

### E. Mitochondrial Isolation and Respiration Buffers

In certain embodiments, methods, uses, and compositions described herein utilize isolation and respiration/storing buffers for mitochondria. In certain embodiments, mitochondrial isolation and respiration/storing buffers used are described in International (PCT) Patent Application No. PCT/US2020/047359, filed Aug. 21, 2020, which is incorporated by reference in its entirety.

In certain embodiments, a mitochondrial isolation buffer (e.g., for isolation of mitochondria) comprises a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an agent that acts as a membrane stabilizer and/or oxygen radical scavenger and/or binder of Ca²⁺ and/or binder of free fatty acid (e.g., bovine serum albumin, BSA); and a serine protease inhibitor (e.g., phenylmethylsulfonyl fluoride (PMFS), also called phenylmethane sulfonyl fluoride). In certain embodiments, the composition further comprises isolated donor mitochondria, e.g., fibroblast mitochondria, MSC mitochondria.

In certain embodiments, a mitochondrial isolation buffer is comprised of 300 mM sucrose, 10 mM K-HEPES, 1 mM K-EGTA, 0.1% BSA and 0.25 mM PMSF (Sigma Aldrich, St Louis, MO, USA). In certain embodiments, the osmolarity of a buffer is about 325 mOsm. In certain embodiments, the concentration of potassium ion is 11 mM. Bovine serum albumin (BSA) is a membrane stabilizer, oxygen radical scavenger, and binds Ca²⁺ and free fatty acids. Phenylmethylsulfonyl fluoride (PMSF), also called phenylmethane sulfonyl fluoride, is a serine protease inhibitor used in the preparation of cell lysates. Lysosomes are organelles that contain digestive enzymes which digest excess or worn-out organelles. During the procedure of cell homogenization (e.g., to obtain mitochondria), some lysosomes may be damaged and release the digestive enzymes to the cell lysate. In certain embodiments, in order to prevent the damage of mitochondria from digestive enzymes, PMSF can be included in an isolation buffer. In certain embodiments, an isolation buffer does not contain antibiotics.

In certain embodiments, a mitochondrial respiration buffer is administered to a subject and/or used to maintain isolated mitochondria stably in solution. In certain embodiments, a respiration buffer comprises one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)] [e.g., monopotassium phosphate (KH₂PO₄)]; a source of magnesium ion [e.g., magnesium chloride (MgCl₂)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an antioxidant [e.g., taurine]; a cytoprotective agent that binds to calcium ion [e.g., lactobionate or salt thereof, e.g., K-lactobionate]; and an agent that acts as a membrane stabilizer and/or oxygen radical scavenger and/or binder of Ca²⁺ and/or binder of free fatty acid (e.g., bovine serum albumin, BSA). In certain embodiments, a composition further comprises isolated donor mitochondria (e.g., fibroblast mitochondria, MSC mitochondria). In certain embodiments, a respiration buffer does not contain antibiotics.

In certain embodiments, a mitochondrial storing buffer is comprised of 240 mM sucrose, 2 mM KH₂PO₄, 3 mM MgCl₂, 10 mM K-HEPES, 20 mM Taurine, 1 mM K-EGTA, 0.1% BSA and 15 mM K-lactobionate (Sigma Aldrich, St Louis, MO, USA). Taurine acts as an antioxidant that scavenges free radical species generated by mitochondria, and is also involved to membrane stabilization, osmoregulation and ion channel regulation. Lactobionate has cytoprotective property and prevents mitochondrial swelling. Lactobionate also binds to calcium ion with high affinity and acts as a calcium chelator. In certain embodiments, the osmolarity of a mitochondrial storing buffer is about 325 mOsm. In certain embodiments, a mitochondrial storing buffer contains about 28 mM potassium ion. In certain embodiments, buffers are sterilized by filtering (e.g., through a 0.22 μm filter), aliquoted to small vials and stored at −80° C.

In certain embodiments (e.g., clinical uses discussed herein), mitochondria in a storing buffer is administered (e.g., intramuscularly, intravenously, subcutaneously) (e.g., both intramuscularly and intravenously) to a human subject. High potassium ion concentrations are dangerous for injection to humans (e.g., 91 mM potassium ion concentration). In certain embodiments, a reduced concentration of all K⁺ salts is used in a mitochondrial respiration buffer (e.g., 2 mM KH₂PO₄, 10 mM K-HEPES, and 15 mM K-lactobionate). In certain embodiments, the final concentration of K⁺ in a respiration buffer solution used is similar to clinical intravenous solution with potassium chloride (e.g., about 28 mEq) (e.g., from 20 mEq to 40 mEq). To keep the osmolarity of a respiration buffer at a desired level, the concentration of sucrose can be increased in the respiration buffer. In certain embodiments, a respiration buffer is not administered along with antibiotics.

### EXEMPLIFICATION

### Isolation and Preservation of Mitochondria From Human Tissue

The present experimental example describes methods for the isolation and preservation of mitochondria from human fibroblasts and human mesenchymal stromal/stem cells (MSCs) derived from bone marrow. Among other things, the results demonstrate isolated mitochondria from cryopreserved MSCs are functional and useful in MOT.

### Fibroblast, Mesenchymal Stromal Cell (MSC) and NSC34 Cell Expansion

Human primary fibroblasts were established and stored in liquid nitrogen according to methods described in International (PCT) Patent Application No. PCT/US2020/047359, filed Aug. 21, 2020, and published as International Publication No. WO 2021/141637. Human primary fibroblasts were recovered from liquid nitrogen and cultured in alpha MEM (GIBCO, Carlsbad, CA, USA) containing 5% human platelet lysate (HPL) (Mill Creek Life Sciences, Rochester, MN, USA). Human MSCs were derived from bone marrow and obtained from RoosterBio (Frederick, MD, USA). MSCs were recovered from liquid nitrogen and cultured in the complete Rooster media (Frederick, MD, USA). NSC-34, a hybrid cell line, was produced by fusion of motor neuron enriched, embryonic mouse spinal cord cells with mouse neuroblastoma. NSC-34 was purchased from Cedarlane corporation (Ontario, Canada) and cultured in Dulbecco's modified eagle medium (DMEM) (GIBCO, Carlsbad, CA, USA) containing 10% fetal bovine serum (FBS). When cells grew to 80% confluence in a flask, they were exposed to TrypLE expression solution (GIBCO, Carlsbad, CA, USA) for removal from the flask and subsequently sub-cultured at 37° C. and 5% CO₂.

### Mitochondrial Staining With JC-1

MMP generated by proton pumps is an essential component in the process of energy storage during oxidative phosphorylation (OXPHOS). Membrane potential-dependent dyes such as JC-1 (5,5′,6,6′-tetrachloro-1,1′,3,3′-tetraethylbenzimidazolocarbocyanine iodide) and MitoTracker dyes were used to stain mitochondria and monitor mitochondrial potential. For JC-1 staining, mitochondria were stained with mitochondria staining kit (Sigma CS0390, St. Louis, MO, USA). The protocol was referred to the document of the product's manufacturer. Fibroblasts and MSCs were incubated with JC-1 solution for 20 minutes at 37° C. in humidified atmosphere containing 5% CO₂. Fluorescence was observed by Olympus IX83 fluorescent microscope. In cells which maintained electrochemical potential gradient, the dye concentrates in mitochondria, where it formed bright red fluorescent aggregates (J-aggregates). If cells failed to maintain MMP, the JC-1 was dispersed through the entire cells resulting in a shift from red to green fluorescence (JC-1 monomers). Cells were treated with mitochondrial inhibitors, valinomycin or FCCP (Carbonyl cyanide-4 (trifluoromethoxy) phenylhydrazone). The treated cells were controls of mitochondrial dissipation.

Isolation of mitochondria

Fibroblasts or MSCs were centrifuged for 5 minutes at 400 g and at 4° C. to remove the media. Cell pellet was re-suspended in ice-cold 300 mM sucrose mitochondrial isolation buffer (MIB) (Sigma Aldrich, St. Louis, MO, USA) and homogenized by bead beating (Bead Ruptor 12, Omni International homogenizer company, Kennesaw, GA, USA). The cell lysate was centrifuged for 10 minutes at 700 g and at 4° C. Then the supernatant was transferred to new centrifugation tubes and centrifuged for 10 minutes at 9,000 g at 4° C. The supernatant was removed. The wet weight of mitochondria was measured. The mitochondrial pellet was re-suspended with 240 mM sucrose mitochondrial respiration buffer (MRB) (Sigma Aldrich, St. Louis, MO, USA). All reagents used in experimental procedures were sterile. Mitochondrial suspension was cooled in wet ice. MMP and mitochondrial ATP content were measured as described below.

### Measurement of MMP

A stock solution of JC-1 was added to mitochondrial suspension to a final concentration 1 μg/ml. The mixture was incubated for 10 minutes at room temperature. Isolated mitochondria were treated with mitochondrial-inhibitor valinomycin. The treated cells were controls of mitochondrial dissipation. Red fluorescent J-aggregates in intact mitochondria could be observed under fluorescent microscope. The relative fluorescence units (RFU) could be read in multiple plate fluorimeter using end-point method with the setting of Ex/Em: 490 nm/590 nm.

### Measurement of ATP Content in Cells and Isolated Mitochondria

ATP content was measured with ATPlite kit (Perkin Elmer Inc., Waltham, MA, USA). A detailed procedure was followed as provided for by the product manual. In brief, the method was conducted using the following protocol: (1) 50 μl of mammalian cell lysis solution was added to 100 μl MRB per well in a 96-well plate with white wells and a clear bottom, (2) the plate was shaken for 5 minutes, (3) 50 μl substrate solution was added to all wells and shaken the plate for 5 minutes, (4) the luminescence of the plate was measured, and (5) the ATP content of samples was calculated using the ATP standard curve.

### MOT of NSC-34 Cells With Mitochondria

Cryogenically frozen MSCs were quickly thawed (<1 minute) by gently swirling the vials in the 37° C. water bath. Mitochondria of the recovered MSCs or fresh MSCs were labelled using 150 nM of MitoTracker Red dye (Thermofisher Scientific, Waltham, MA, USA) at 37° C. and 5% CO₂ for 30 minutes. The cells were washed with Hanks' balanced salt solution (HBSS) 3 times to remove the dye. Then, mitochondria were isolated. The MitoTracker Red-labelled mitochondria were added to NSC-34 cells that grew on glass surface in glass-bottom culture dishes and incubated overnight at 37° C. and 5% CO₂. Then the NSC-34 cells were washed 3 times with pre-warmed HBSS to remove the labelled mitochondria in the media. The cell dishes were observed under fluorescent microscope.

### Statistical Analysis

Student's t-test was used to test statistical significance. A p-value less than 0.05 was judged to be of statistical significance.

### Results

### Cryopreserved Fibroblasts and MSCs Maintain MMP.

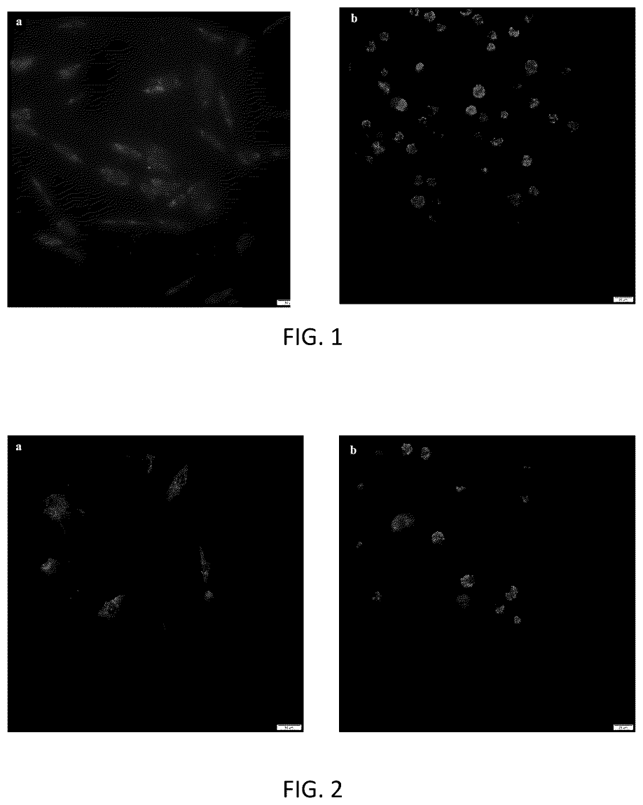

Cryovials containing frozen fibroblasts or MSCs were removed from liquid nitrogen storage and immediately placed into a 37° C. water bath. Cells were quickly thawed (<1 minute) by gently swirling the vials in the 37° C. water bath until there was just a small bit of ice left in the vials. The vials were transferred to a laminar flow hood. MMP was measured by JC-1 staining. Red fluorescence was observed in mitochondria of the fibroblasts and MSCs. Red fluorescent brightness and distribution in the cryopreserved cells were not different from the growing fibroblasts and MSCs that attached on surface of slides (FIGS. 1 and 2). Valinomycin or FCCP-treated cells lost most red fluorescence. The data show that cryogenically frozen fibroblasts and MSCs in liquid nitrogen maintain similar MMP to the fresh fibroblasts and MSCs.

FIG. 1 is a series of fluorescent images (FIG. 1, panels a and b) comparing the mitochondrial membrane potential (MMP) of mitochondria from fresh fibroblasts (FIG. 1, panel a) with cryopreserved fibroblasts (FIG. 1, panel b). Mitochondria were stained using membrane potential dependent dye JC-1. In FIG. 1, cryopreserved fibroblasts are shown to have a MMP that is comparable to fresh fibroblasts.

FIG. 2 is a series of fluorescent images (FIG. 2, panels a and b) comparing the mitochondrial membrane potential (MMP) of mitochondria from cryopreserved MSCs (FIG. 2, panel b) to fresh MSCs (FIG. 2, panel a). Mitochondria were stained using membrane potential dependent dye JC-1. In FIG. 2, cryopreserved MSCs have a MMP that is comparable to fresh MSCs.

### Isolated Mitochondria From Cryopreserved Cells Maintain MMP and ATP Content.

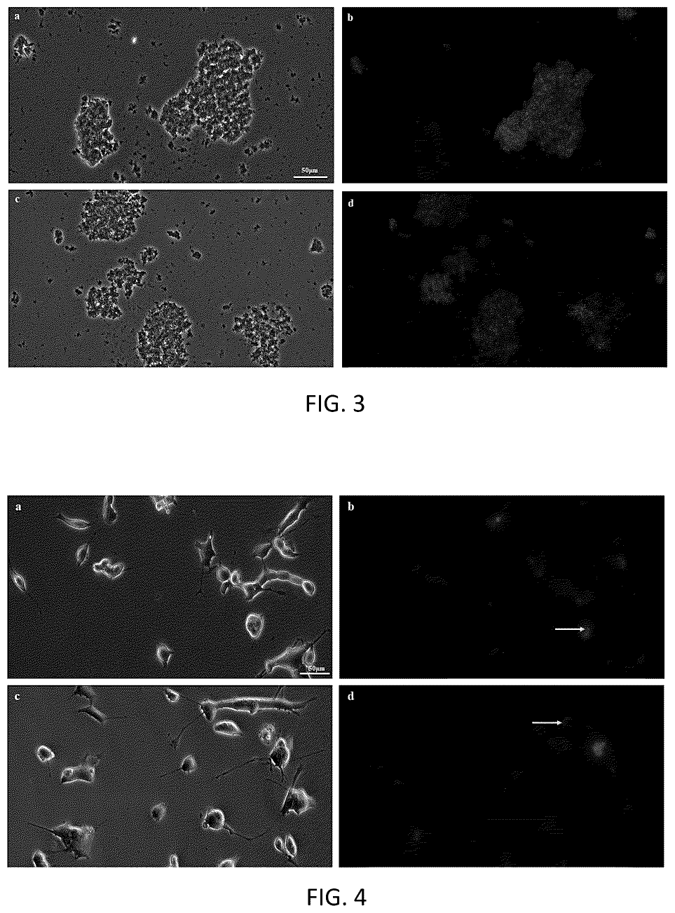

Vials containing 50×10⁶ frozen MSCs in liquid nitrogen or dry ice were immediately placed into a 37° C. water bath by gently swirling the vials until there was just a small bit of ice left in the vials. Mitochondria were isolated from the MSCs and proceeded to measurement of MMP and ATP content. The mitochondria from the cryopreserved MSCs actively took up dye JC-1 and formed bright red fluorescence (J-aggregates) that was similar to the mitochondria isolated fresh MSCs (FIG. 3). In mitochondrial suspension with concentration 100 mg/ml, RFU of the mitochondria of cryopreserved MSCs (435±45) is slightly lower than the mitochondria from fresh MSCs (488±82), but the difference is not significant (p>0.05).

In agreement with MMP, ATP content of the mitochondria from frozen MSCs (51.8±7.7 pmol/mg mitochondria) is slightly less than the mitochondria from fresh MSCs (64.8±8.9 pmol/ml), but the difference is not significant (p>0.05). These results of MMP and ATP content may demonstrate that cryopreservation maintains mitochondrial function of MSCs.

FIG. 3 shows panels of fluorescent and phase contrast images of MSCs. The images show that mitochondria of cryopreserved MSCs maintain comparable MMP to mitochondria from fresh MSCs. FIG. 3, panel a is a phase contrast image of mitochondria from fresh MSCs. FIG. 3, panel b is a fluorescent image of mitochondria of fresh MSCs. FIG. 3, panel c is a phase contrast image of mitochondria from cryopreserved MSCs. FIG. 3, panel d is a fluorescent image of mitochondria of cryopreserved MSCs.

### Mitochondria Isolated From Cryopreserved MSCs Transfer Into NSC34 Cells.

Mitochondria isolated from MSCs were labeled with MitoTracker Red and co-cultured with NSC-34 cells for 16 hours. After 16 hours, the mitochondria were seen within the NSC-34 cells when using a fluorescent microscope. Mitochondria from both fresh and cryopreserved MSCs could enter to NSC-34 cells (FIG. 4). FIG. 4 shows a series of images of mitochondria isolated from fresh and frozen MSCs transferred into NSC-34 cells. FIG. 4, panels a and b show phase contrast and fluorescent images, respectively, of NSC-34 cells co-cultured with the mitochondria of fresh MSCs. FIG. 4, panels c and d show phase contrast and fluorescent images of NSC-34 cells, respectively, co-cultured with the mitochondria of cryopreserved MSCs. Arrows in FIG. 4, panel b and panel c show MitoTracker Red labelled mitochondria of MSCs within NSC-34 cells.

The results from the images show that mitochondria from cryopreserved MSCs may have similar abilities to enter into NSC-34 cells as compared with mitochondria from fresh MSCs.

### Fibroblasts and MSC Characteristics

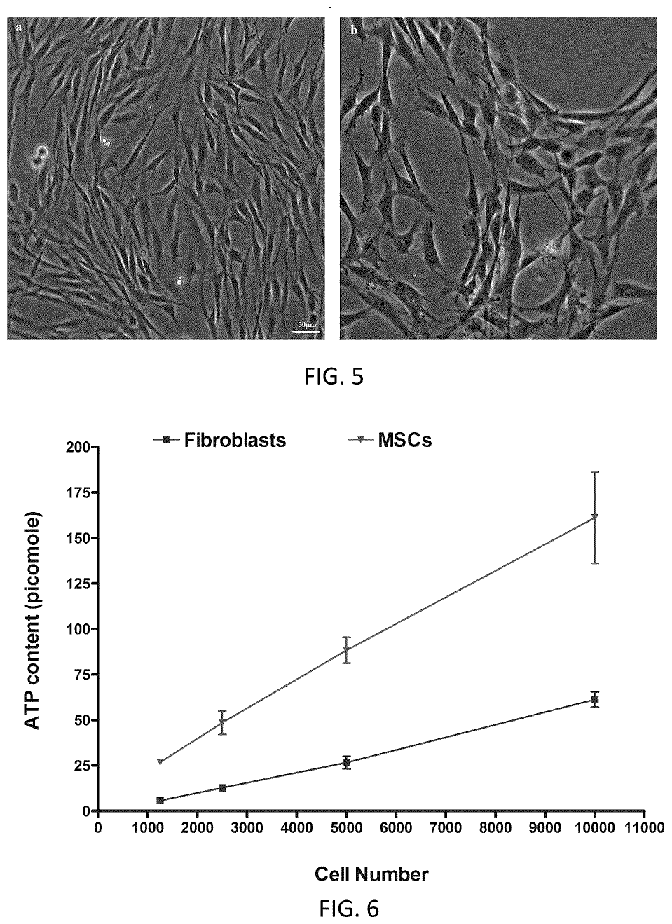

Fibroblasts and MSCs have similar morphological characteristics (FIG. 5). FIG. 5, panel a shows phase contrast image of fibroblasts. FIG. 5, panel b shows a phase contrast image of MSCs. Both fibroblasts and MSCs have abundant mitochondria as shown with the membrane potential dependent dye JC-1 in FIGS. 1 and 2. However, MSCs produce higher levels of ATP than fibroblasts (all p<0.01, at wells with cell amount 1250, 2500, 5000 and 10000 per wells) (FIG. 6). In FIG. 6, cells were grown on 96-well cell culture plates at 37° C. overnight. The details of the culture methods are provided herein.

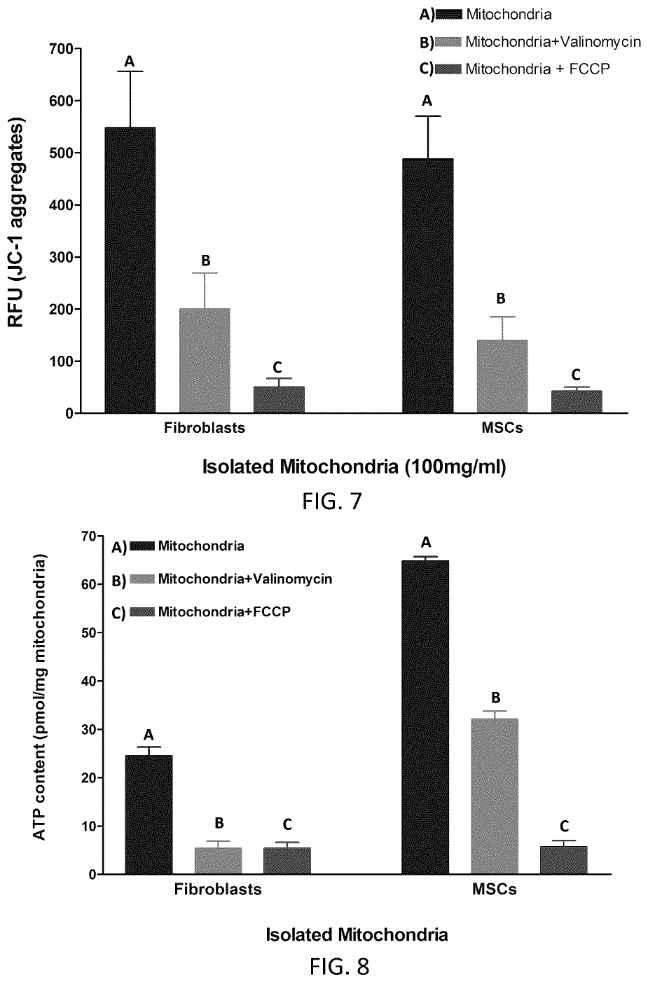

Further, MMP and ATP content of mitochondria isolated from fresh fibroblasts and MSCs were measured as shown in FIGS. 7 and 8. FIG. 7 shows the MMP of mitochondria isolated from fibroblasts and MSCs. The relative fluorescence units (RFU) of the mitochondria of fibroblasts and MSCs are not significantly different (p>0.05). Valinomycin or FCCP-treated mitochondria were used as the controls of mitochondrial dissipation. Details regarding the treatments are provided herein. In brief, valinomycin and FCCP are mitochondrial inhibitors.

MMP was not significantly different between the isolated mitochondria of fibroblasts (RFU: 548±108) and MSCs (RFU: 488±82) (p>0.05) (FIG. 7). In alignment with the ATP content results from whole fibroblasts and MSCs, isolated mitochondria from MSCs (64.8±8.9 pmol/mg mitochondria) have significantly higher ATP content than the mitochondria of fibroblasts (24.5±1.8 pmol/ml) (p<0.01) (FIG. 8). In FIG. 8, the ATP content of mitochondria isolated from fibroblasts and MSCs is shown. The ATP content of the mitochondria from MSCs is significantly higher than the mitochondria of fibroblasts (p<0.01), despite having similar amounts of mitochondria.

### Discussion

Without wishing to be bound to any particular theory, the results provided herein demonstrate MOT may replenish mitochondria and mtDNA and may restore or improve mitochondrial function of defective cells. Moreover, MOT may yield positive therapeutic results in animal models of disease, as demonstrated in rabbit models of cardiac ischemia-reperfusion, rat models of Parkinson's disease (PD), rat models of brain ischemia, spinal cord injury (SCI), and traumatic brain injury (TBI). Also, MOT clinical trials for acute respiratory distress syndrome, myocardial ischemia, ischemia-reperfusion injury and infertility have shown MOT can confer benefits onto patients. However, one of the major challenges for MOT is mitochondrial preservation. The shelf life of isolated mitochondria is short. Mitochondria isolated from MSCs lost 70% MMP and 40% ATP content after 2 days of storage at 4° C. Long term cold storage and cryopreservation of isolated mitochondria has not been successful as cold storage and cryopreservation lead to a decrease in respiratory capacity and damage to mitochondrial membrane structures over time. Therefore, routine MOT procedures have typically been performed by injection of mitochondria to patients as soon as possible after performing a series of steps, which can involve spending several weeks expanding cells. Previous methods of MOT production had several disadvantages including: (1) requiring an expensive cGMP compliant facility for cell expansion and process in clinical facilities; (2) requiring several weeks for cell expansion, which is not practicable for urgent/imminent use cases such as ischemic-reperfusion stroke, traumatic brain, and spinal cord injuries; and (3) requiring difficult-to-coordinate scheduling of cell processes and patient needs.

Cryogenic banking and shipping of cells with cryoprotectant (e.g., cryopreservation media) has removed the need for continuous culture. Continuous culture of cells results in phenotype drift, as well as consuming large amounts of resources (e.g., for maintenance alone). Cryogenic methods have been used herein to deliver emerging cell-based therapies. Even though cryopreservation commonly decreases functionality of cells, including mitochondria, and post-thaw viability of cryopreserved cells maintain high (>90%) viability after thawing. In the example herein, the Applicant found that fresh and cryopreserved fibroblasts and MSCs have comparable MMP and mitochondrial ATP content (see, e.g., FIGS. 1-3). Moreover, mitochondria isolated from cryopreserved fibroblasts or MSCs could transfer into NSC-34 neural cells (FIG. 4). The current results demonstrate that cryopreserved cells are feasible mitochondrial donors to deliver MOT for clinical trials.

In order to overcome the disadvantages of routine MOT, which uses fresh cells for mitochondrial donors, the inventors are exploring an enhanced methodology for MOT clinical trials. cGMP fibroblasts or MSCs will be expanded and banked in a central cGMP compliant cell factory. The frozen cells will be cryogenically shipped to multiple clinical sites which have installed BioSpherix Xvivo System X2. The system is a portable ISO Class 5 closed aseptic isolator and designed for producing and processing cells in compliance with regulatory GMPs. Mitochondria will be isolated from the cryopreserved cells in the Xvivo System X2. The isolated mitochondria will be injected to patients after isolation as soon as possible.

We reported a case of MOT study by using mitochondria from human fibroblasts. The MOT improved leg muscle strength and recovered all sensory sensation of legs in a patient who suffered from desperate amyotrophic lateral Sclerosis (ALS). Fibroblasts express similar cell surface markers to MSCs (positive for CD73, CD90 and CD105 and negative for CD14, CD34, CD45, CD19 and HLA-DR). In this study, the inventors have tested whether MSCs mitochondria are comparable to fibroblast mitochondria. We find that MMP of fibroblasts and MSCs are comparable. However, mitochondria isolated from MSCs produce higher ATP content than mitochondria isolated from fibroblasts.

In summary, mitochondria of fresh and cryopreserved fibroblasts and MSCs are comparable. Cryopreserved fibroblast and MSCs are alternative mitochondrial donors for MOT to fresh cells. In addition, MSCs could be a source of higher ATP-producing mitochondria than mitochondria derived from fibroblasts.

### EXEMPLARY EMBODIMENTS

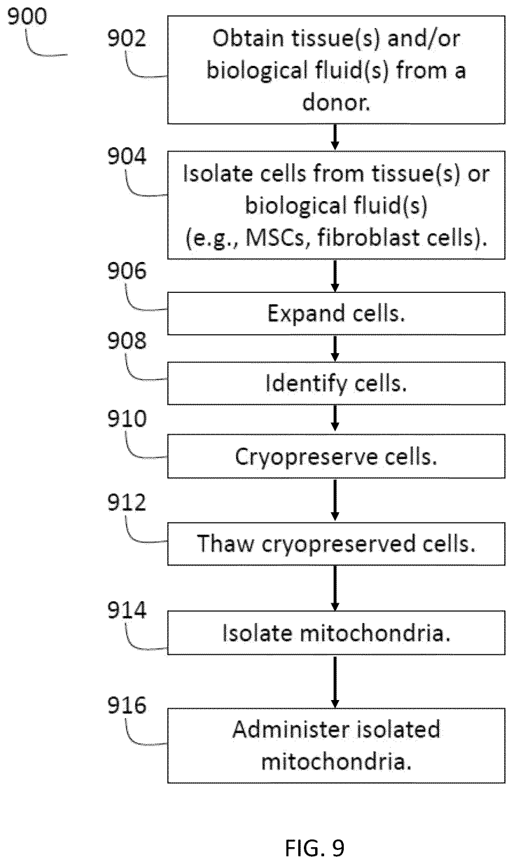

FIG. 9 shows an illustrative flow diagram of an embodiment of a method (900) as described herein. The figure is not intended to limit the present disclosure.

In some embodiments, a method (900) includes obtaining tissue(s) and/or biological fluid(s) from a donor (e.g., a human donor) (902) as described, for example, in section C above.

In certain embodiments, cells are isolated from tissue(s) and/or biological fluid(s) obtained from a donor (904). Tissue(s) and/or biological fluid(s) can be selected based on a cell type(s) being isolated from the tissue(s) and/or the biological fluid(s). In certain embodiments, tissue(s) and/or biological fluid(s) are selected as further described, for example, in section C above. In certain embodiments as described herein, tissues and/or biological fluids are obtained from a donor without a neurodegenerative disease or other condition that would affect mitochondria function (e.g., MMP, ATP content, mitochondrial respiration, amount of mitochondria, etc.) or structure as described in, for example, section C above.

In some embodiments, isolated cells (e.g., primary cells from a human donor, e.g., MSCs, fibroblast cells) are expanded (906) (e.g., after isolation) as described in, for example, section C above. In some embodiments, expansion of cells does not use antibiotics. In some embodiments, during cell expansion, cells are induced (e.g., stimulated) to increase an amount of (e.g., a number of, a concentration of) mitochondria within the cells.

In some embodiments, cells (e.g., primary cells) from a donor are identified (e.g., characterized) as having characteristics corresponding to a desired phenotype (908) as described in, for example, section C above. For example, cells can be identified after isolation from tissue(s) and/or biological fluid(s) (904), after and/or during expansion (906), prior to and/or after cryopreservation (910). In some embodiments, cells are identified during, before, or after more than one step (e.g., to ensure against phenotypic drift). For example, in some embodiments, identifying cells (908) may be performed as part of isolating cells (e.g., for example as with flow cytometry) to select cells having one or more characteristics corresponding to a desired phenotype.

In some embodiments, cells are subsequently cryopreserved (910) (e.g., in a cryopreservation medium). Cryopreserved cells can be transported or stored for an extended period of time under appropriate conditions. In some embodiments, cryopreserved cells are stored at a temperature of about −60° C. or less (e.g., about −70° C. or less, about −80° C. or less, about −100° C. or less, about −120° C. or less, about −135° C. or less) (e.g., on dry ice, using liquid nitrogen).

In some embodiments, cryopreserved cells are thawed (912) (e.g., in a fluid bath, e.g., a water bath) at a temperature of about 20° C. to about 40° C. (e.g., about 37° C.). For example, in some embodiments, a vial containing cryopreserved cells is thawed until there is a small amount of ice left in the vial.

In some embodiments, mitochondria are isolated (914) from cryopreserved cells after (e.g., immediately after) being thawed (912) using methods described herein. For example, in some embodiments, cells are not expanded or cultured after thawing and prior to isolation of mitochondria. In some embodiments, mitochondria is isolated using differential centrifugation. In some embodiments, to isolate mitochondria, cells are centrifuged to remove cryopreservation media, resulting in a cell pellet. In certain embodiments, a cell pellet and/or mitochondria are suspended in a buffer (e.g., a mitochondrial isolation buffer (MIB), e.g., as described herein in section E). In some embodiments, thawed cells are lysed (e.g., by homogenization, e.g., bead-beating) to release contents of the cells. In some embodiments, cell lysate is centrifuged to obtain mitochondria from a supernatant containing mitochondria. In some embodiments, mitochondria are suspended in a mitochondrial respiration buffer (MRB) (e.g., a MRB described herein in section E).

In some embodiments, steps of the method (900), such as thawing cryopreserved cells (912) and isolating mitochondria (914), are performed in an aseptic environment (e.g., an environment substantially free from contaminants, e.g., in a Xvivo System model X2).

In some embodiments, isolated mitochondria (e.g., a composition comprising mitochondria) are subsequently administered to a subject (916). In some embodiments, isolated mitochondria (e.g., a composition comprising isolated mitochondria) can be stored for at least one hour (e.g., at least 2 hours, at least 3 hours, at least about 6 hours, e.g., at least about 12 hours, e.g., at least about 24 hours, e.g., at least about 48 hours, e.g., at least about 5 days) prior to administration. In some embodiments, isolated mitochondria are administered to a subject within 2 days of identification of a condition (e.g., a disease, e.g., a disease related to mitochondrial dysfunction), an injury, or a symptom (e.g., a symptom related to mitochondrial dysfunction) (e.g., within 24 hours, within 12 hours, within 6 hours, within 3hours, within 2 hours, or within 1 hour of said identification). In some embodiments, a subject has an acute condition (e.g., an acute injury, a sudden worsening and/or a sudden presentation of a disease, etc.). In some embodiments, a subject is suffering from a disease and/or condition described in, for example, sections A or B. In some embodiments, diseases and conditions include those described in PCT/US20/47359, PCT/US23/32292, or PCT/US23/32294, each of which are incorporated by reference in their entireties.

In some embodiments, isolated mitochondria is added to a composition comprising extracellular vesicles (EVs) (e.g., after isolating the mitochondria) (e.g., prior to administration) to create an EV-mitochondria composition (e.g., for administration to a subject). EV-mitochondria compositions and corresponding methods are described in, for example, U.S. Provisional Application No. 63/455,397, filed Mar. 29, 2023, and U.S. Provisional Application No. 63/604,044, filed Nov. 29, 2023, each of which are incorporated by reference in their entireties. In some embodiments, the extracellular vesicles comprise one or more members selected from the group consisting of: (i) microvesicles (MVs) (e.g., ranging from about 100 nm to about 1 micrometer in diameter, e.g., comprising cytosolic and plasma membrane associated proteins), exosomes, and apoptotic bodies; (ii) microvesicles (MVs) (e.g., ranging from about 30 nm to about 150 nm in diameter, e.g., formed by an endosomal route); and (iii) apoptotic bodies (e.g., ranging from about 50 nm up to about 5 micrometers in diameter, e.g., comprising intact organelles and/or chromatin and/or glycosylated proteins). In some embodiments, extracellular vesicles comprise extracellular vesicles of mesenchymal stromal cells (imEVs). In some embodiments, EV-mitochondria compositions comprises a mixture of mitochondria and EVs in a ratio from about 1:50 (mitochondria: EVs, in vol.) to about 50:1 (mitochondria: EVs, in vol.) [e.g., wherein the ratio is from about 2:1 to about 50:1, or wherein the ratio is from about 5:1 to about 15:1, or wherein the ratio is about 9:1]. In some embodiments, mitochondria accumulate within structures formed by the EVs in the EV-mitochondria composition. In some embodiments, EV-mitochondrial compositions improves preservation of mitochondrial membrane potential (MMP) of isolated mitochondria (e.g., using extracellular vesicles to improve preservation of MMP of isolated mitochondria and/or to improve preservation/retention of mitochondrial adenosine triphosphate (ATP) content) (e.g., using cryopreserved cells to improve preservation of MMP of isolated mitochondria and/or to improve preservation/retention of mitochondrial adenosine triphosphate (ATP) content).

In some embodiments, methods described herein include mitochondria being administered to a subject along with (or without) one or more other compositions and/or compounds. For example, in some embodiments, mitochondria are administered to a subject (e.g., as described therein) being treated with a pharmaceutical agent (e.g., hydroxychloroquine and/or chloroquine) for indications accompanied by high Reactive Oxygen Species (ROS). In some embodiments, a subject is not administered an antibiotic. In some embodiments, a subject is administered one or more drugs and/or adjuvants. In some embodiments, a subject is administered an iron-chelating agent (e.g., desferrioxamine or deferasirox). In some embodiments, a subject is administered an antioxidant and/or a probiotic. In some embodiments, a subject is administered a composition comprising isolated mitochondria and a pharmaceutically acceptable carrier (e.g., as described herein).

### OTHER EMBODIMENTS

While we have described a number of embodiments, it is apparent that our basic disclosure and examples may provide other embodiments that utilize or are encompassed by the compositions, kits, and methods described herein. Therefore, it will be appreciated that the scope of is to be defined by that which may be understood from the disclosure and the appended claims rather than by the specific embodiments that have been represented by way of example.

All references cited herein are hereby incorporated by reference.

1. A method of isolating mitochondria from cryopreserved cells, the method comprising:
thawing cryopreserved frozen primary cells; and
isolating the mitochondria from the thawed cells.

2. The method of claim 1, wherein the method further comprises administering a composition comprising the isolated mitochondria to a human subject.

3. The method of claim 1, wherein the primary cells comprise mesenchymal stromal cells (MSCs).

4.-5. (canceled)

6. The method of claim 1, wherein the primary cells comprise fibroblast cells.

7. (canceled)

8. The method of claim 1, wherein the cryopreserved frozen primary cells are stored in a cryopreservation media that does not comprise antibiotics.

9.-12. (canceled)

13. The method of claim 1, wherein the method comprises isolating the primary cells from tissues(s) or biological fluids of a human donor and expanding the isolated cells.

14.-24. (canceled)

25. The method of claim 1, wherein the method comprises identifying primary cells comprising mitochondria characterized as having high adenosine triphosphate (ATP) content.

26.-29. (canceled)

30. The method of claim 1, wherein the method comprises suspending the isolated mitochondria in a mitochondrial respiration buffer (MRB).

31. The method of claim 30, wherein the mitochondrial respiration buffer (MRB) comprises:
a buffering agent;
a chelating agent;
a sugar;
an agent that acts as a membrane stabilizer and/or oxygen radical scavenger and/or binder of Ca²⁺ and/or binder of free fatty acid; and
a serine protease inhibitor.

32. (canceled)

33. The method of claim 1, wherein the method comprises lysing the thawed primary cells.

34. The method of claim 1, wherein the method does not comprise culturing and/or expanding the thawed primary cells.

35.-48. (canceled)

49. The method of claim 1, wherein the method comprises adding the isolated mitochondria to a composition comprising extracellular vesicles (EVs) to create an EV-mitochondria composition.

50. The method of claim 49, wherein the extracellular vesicles comprise one or more members selected from the group consisting of:
(i) microvesicles (MVs), exosomes, and apoptotic bodies;
(ii) microvesicles (MVs); and
(iii) apoptotic bodies.

51.-56. (canceled)

57. A kit comprising the isolated mitochondria produced by the method of claim 1.

58. A composition comprising the isolated mitochondria produced by the method of claim 1.

59. A composition comprising isolated mitochondria characterized as having high ATP content.

60. (canceled)

61. The composition of claim 59, wherein the composition comprises:
a buffering agent;
a chelating agent;
a sugar;
an agent that acts as a membrane stabilizer and/or oxygen radical scavenger and/or binder of Ca²⁺ and/or binder of free fatty acid; and
a serine protease inhibitor.

62.-75. (canceled)

76. A method for transplantation of mitochondria in a subject for treatment and/or prevention and/or amelioration of an acute injury and/or a condition associated with mitochondrial damage, the method comprising administering to said subject mitochondria isolated from cryopreserved primary cells of a donor to treat and/or prevent and/or ameliorate said injury and/or said condition.

77. A composition comprising isolated donor mitochondria for use in the manufacture of a medicament for transplantation of mitochondria in a human subject, wherein the donor mitochondria are isolated from cryopreserved primary cells of a human donor, and wherein the isolated mitochondria are characterized as having a high ATP content.

78. (canceled)

79. A kit comprising cryopreserved primary cells in a sufficient quantity to obtain isolated mitochondria in a unit dosage effective to treat and/or prevent and/or ameliorate an acute injury, and/or a disease associated with mitochondrial damage, the kit comprising:
cryopreserved primary cells of a donor; and
a mitochondrial respiration buffer (MRB).

80.-91. (canceled)
